(19) United States
(12) Patent Application Publication
(10) Pub. No.: US 2013/0266124 A1
(43) Pub. Date: Oct. 10, 2013

(54) Magnification Marker for Radiography

(71) Applicant: Nicholas B. Coursolle, Portland, OR (US)

(72) Inventor: Nicholas B. Coursolle, Portland, OR (US)

(21) Appl. No.: 13/628,326

(22) Filed: Sep. 27, 2012

Related U.S. Application Data
(60) Provisional application No. 61/540,634, filed on Sep. 29, 2011.

(51) Int. Cl. A61B 19/00

(52) U.S. Cl. CPC A61B 19/54
USPC 378/163

(57) ABSTRACT

An apparatus for a securing a magnification marker to a subject such as a human patient includes an elongated strap that has a length sufficient for surrounding the subject around a region, such as a knee or pelvis, that is to be radiographed. A radiopaque magnification marker of a predetermined size is carried by the strap. Fasteners are included for fastening the strap to itself with the strap in a fastened configuration surrounding the subject, thereby to secure the strap to the subject with the magnification marker adjacent to the region to be radiographed. In one embodiment, the strap is stretchable and configured to allow the fastened strap to slide along the subject to adjust the position of the magnification marker relative to the region.

### BACKGROUND AND SUMMARY

This invention relates to an apparatus and method for quick and precise positioning of a magnification marker for radiography.

Radiographic images are critically important for orthopedic surgeons in preparing for joint repair or replacement surgery. The images may be prepared using any of a number of techniques, including conventional X-rays (using X-ray source and radiation-sensitive film) as well as more recently popular Digital Radiography and Computed Radiography.

The radiographic images are particularly useful in selecting the proper size of a prosthetic device (such as an artificial hip or knee implant) for a given patient. To this end, a procedure often referred to as X-ray templating has become popular. The templating is carried out on a computer, which displays results and options to a surgeon. The radiograph of a joint may be displayed, for example, is displayed, and the surgeon is provided with several digital images of an implant, each image representing a different size or type of the implant. The surgeon can select (using a touch screen, for example) a particular implant image, and the templating software then generates a composite image of the implant and joint for inspection and manipulation by the surgeon to determine the best size and type of implant prior to surgery.

Radiography generally involves directing the X-Ray beam through one side of an anatomical feature, such as a hip joint, and capturing the unabsorbed parts of the beam on a detector located on the opposite side of the joint. The detector may be conventional film or other X-ray sensitive material such as an array of semiconductor diodes. The detected image reveals radiopaque or nearly opaque areas where the X-rays are nearly fully absorbed (bone) as well as more transparent areas (soft tissue).

Radiography inherently magnifies the size of the structure being imaged, such as a hip joint. This magnification is attributable to the typical divergence of the source X-ray beam. The amount of magnification depends upon the distance between the source and the joint, as well as the distance between the joint and the detector. These distances (hence the amount of magnification) can vary significantly from one radiograph to another.

The present invention is directed to a magnification marker apparatus that can be easily and quickly secured to a patient in the region that is to be radiographed. The magnification marker is radiopaque and spherical and has a known diameter. The marker will thus appear on the radiographic image. The templating software includes an interactive feature that allows the user to precisely mark the diameter on the image and enter into the program the known diameter of the magnification marker. Based on this information, the templating software can establish the precise scale of the radiographic image and conform the scale of the selected implant image (also having a known size) to greatly enhance the accuracy of the composite joint/implant image and thus increase the accuracy in selecting the proper size and type of implant.

### BRIEF DESCRIPTION OF THE DRAWINGS

FIG. 1 is a front side view of a magnification marker apparatus made in accordance with the present invention.

FIG. 2 is a top view of the apparatus shown about to be secured to a subject (shown in cross section) with the magnification marker located in a region that is to be radiographed.

FIG. 3 is a top view of the apparatus shown secured to the subject with the magnification marker in the region that is to be radiographed.

### DETAILED DESCRIPTION

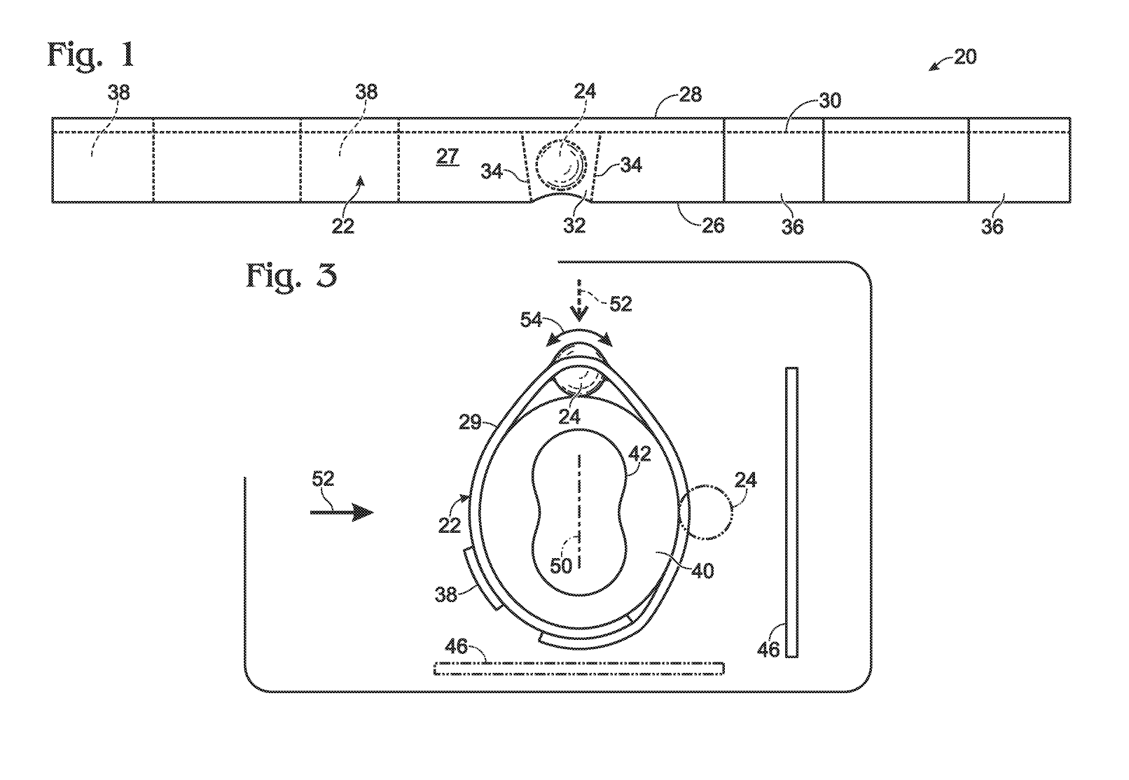

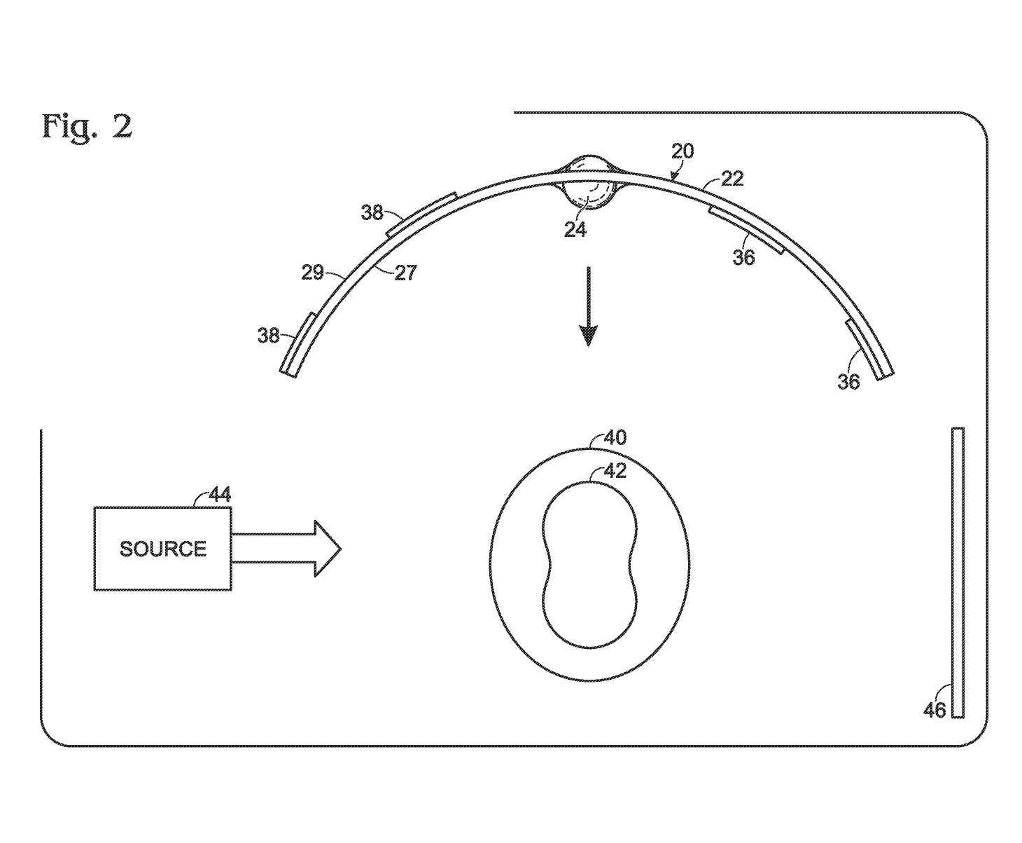

With reference to FIGS. 1 and 2, the magnification marker apparatus 20 (hereafter, “apparatus”) of the present invention includes an elongated strap 22 that carries a spherical magnification marker 24. The strap 22 is fastened to a subject, such as a human patient, so that the magnification marker 24 is adjacent to the region of the subject (a knee joint, for instance) that is to be radiographed.

In one preferred embodiment, the strap 22 is formed of slightly stretchable material, such as 3.0 mm thick neoprene. The material is folded on one long side 26 (FIG. 1) and joined at the opposite long side 28 by stitching 30 as shown in dashed lines. Thus, the strap has a front layer 27 (FIG. 1) and a back layer 29 (FIG. 2).

The magnification marker 24 is a 25.4 mm diameter stainless steel (hence, radiopaque) sphere. In this embodiment of the apparatus 20, the magnification marker 24 is carried at the center of the strap 22 inside of a pocket 32 formed in the strap. Specifically, the magnification marker 24 is between the two strap layers 27, 29 discussed above. The pocket 32 is defined by stitching 34 that joins those two layers on each side of the magnification marker 24. The above-mentioned stitching 30 along the long side 28 of the strap seals the pocket with the magnification marker 24 inside.

Preferably, the pocket 32 is sized so that the magnification marker fits snugly inside and does not move about within the pocket.

The strap 22 includes fasteners 36, 38 for fastening the strap to itself (see FIGS. 2 and 3) with the strap in a fastened configuration surrounding the subject 40, thereby to secure the strap to the subject with the magnification marker 24 adjacent to the region to be radiographed. In the embodiment shown in FIGS. 2 and 3, the subject is the leg 40 of a human patient, and the strap 22 is fastened to the leg to surround the leg 40 to secure the magnification marker 24 adjacent to the region to be radiographed, a knee joint 42.

FIG. 2 also schematically illustrates an exemplary orientation of an X-ray source 44 and detector 46 between which the knee joint 42 is positioned to obtain the radiographic images desired by surgeon.

It is noteworthy the use of radiography, hence the magnification marker apparatus, is not restricted to use with human subjects.

The above-mentioned fasteners 36, 38 are preferably hook-and-loop type. In the embodiment shown in the figures, one end of strap 22 has fixed to it two, spaced apart patches of hook material 36 on the front layer 27 of the strap. At the opposite end of the strap, 22 are fixed two spaced apart patches of loop material 38 on the back layer 29 of the strap. FIG. 3 illustrates the strap 22 applied to the subject 40 with one end of the strap overlapping the other end of the strap so that a pair of patches of hook material 36 and loop material 38 engage at the overlapped part to secure the strap to the subject.

It is contemplated that the hook-and-loop type fastening can be accomplished with various arrangements and sizes of fastener patches applied to the strap. In the preferred embodiment, however, the fastener patches 36, 38 are disposed at discrete, spaced apart locations. This is preferred so that the strap material can be selected to be slightly stretchable and, therefore, able to stretch somewhat between the fastener patches (which are not readily stretchable) to enable the maximum amount of overlap between the engaging hook and loop material patches and thus ensure that the strap is slightly tensed for secure attachment to the subject.

Although the embodiment illustrated shows the strap 22 applied to a knee joint 42, the strap can be sized for used in other anatomical regions. For instance, it is contemplated that the strap 22 could be formed of sufficient length to surround a human pelvis and be secured thereto with the magnification marker adjacent to the particular hip region that is the study of the radiograph. Such a lengthy strap could also be formed with enough fastener patches to allow the same strap to be used for both large (e.g. pelvis) and small (e.g. knee) regions.

With reference to FIG. 3, the magnification marker 24 is securely held adjacent to the knee joint 42 such that the marker is firmly bearing on the outer surface of the leg 40 at the knee. Thus, the marker will not move relative to the region absent a user's manipulation of the apparatus. Moreover, the magnification marker is located on the side of the subject 40 so that it is not between the X-Ray beam (arrow 52) and joint 42 or between the joint 42 and detector 46. Since either of these positions would result in an incomplete image of the joint owing to the presence of the radiopaque marker. For best scaling accuracy, the magnification marker should be located in the plane of the region of interest, in this case, the central plane of the knee joint, as shown by vertical line 50 in FIG. 3.

It is noteworthy that the apparatus 20 of the present invention enables easy adjustment of the position of the magnification marker 24 to meet the positioning requirements mentioned in the preceding paragraph. This adjustment is made by simply rotating the attached strap 22 (see arrow 54) until the magnification marker 24 is properly positioned. This rotation is facilitated by the stretchiness of the strap, which can be tugged and stretched radially outwardly while the strap is rotated. The strap 22 and magnification marker 24 position can be thus adjusted by a relatively small amount to ensure the spherical marker is bisected by the central plane 50 of the joint, or by a larger amount, such as 90 degrees or more, to place the marker 24 in the proper position (dashed lines in FIG. 3) for an image that is taken from an orientation that is rotated 90 degrees from the prior image.

With continued reference to FIG. 3, it will be appreciated that when the strap is adjusted and secured to the subject, the magnification marker 24 is effectively “worn” by the patient. This is quite advantageous in frequent instances where a patient may move or be moved slightly after the radiographed is readied, but before imaging occurs. Since the marker moves with the patient (more precisely, moves with the region of the patient to which it is fastened) there is usually no need to readjust the position of the magnification marker after such patient movement to ensure accuracy. This can reduce the time and effort involved in radiographic procedures.

The strap 22 can be disposable or, preferably, be reusable. Where reuse is desired, the apparatus will include a separate self-adherent wrap material for wrapping the region prior to applying the strap. This will prevent the strap from directly contacting the patient and thus help avoid contamination of the strap. Alternatively, the entire reusable strap may be enclosed within a disposable sleeve that includes exposed fasteners (such as adhesive or hook-and-loop fasteners, snaps etc.) for securing the sleeved strap in place.

To facilitate cleaning or replacement of the strap, the above described pocket 34 may be configured to be openable and closable to allow the magnification marker to be removed and replaced. In this embodiment, the pocket 34 would replace the associated segment of stitching 30 (FIG. 1) with a hook-and-loop fastener or the like to enable the pocket to be opened, to remove the marker, and closed with the marker secured in the pocket.

As an alternative way of properly locating the magnification marker relative to the region that is radiographed, it is contemplated that that the strap may be provided with empty pockets at several locations along the length of the strap. Once the strap is secured to the patient, the user locates the particular pocket that is best oriented to position the magnification marker in the plane of the joint, as described above, and the marker is then placed in the selected pocket.

While the foregoing description was made in the context of preferred embodiments, it is contemplated that modifications to those embodiments may be made without departure from the invention as claimed. For example, the strap need not be formed of stretchable material. Moreover, mating buckle components located on opposing ends of the strap may be used as an alternative or supplemental fastener with at least one buckle component being movable relative to the other for adjusting the circumference of the applied strap. Also, it is contemplated that the marker can be carried on one end of the strap (rather than between ends), which in some cases may make the apparatus easier to manipulate in the course of securing it to the subject. The magnification marker need not be limited to the particular size noted above with respect to the preferred embodiment. In this regard, it is also contemplated the strap or magnification marker or both are provided with radiopaque indicia of the precise size of the magnification marker, thereby revealing the marker size in the radiograph to ensure accuracy in the scaling process.

1. An apparatus for a securing a magnification marker to a subject, comprising:
an elongated strap having a length sufficient for surrounding the subject around a region of the subject that is to be radiographed;
a radiopaque magnification marker having a predetermined size and carried by the strap; and
fasteners for fastening the strap to itself with the strap in a fastened configuration surrounding the subject, thereby to secure the strap to the subject with the magnification marker adjacent to the region to be radiographed.

2. The apparatus of claim 1 wherein the strap is stretchable and configured to allow the fastened strap to slide along the subject to adjust the position of the magnification marker relative to the region.

3. The apparatus of claim 2 wherein the fasteners are disposed on the strap at discrete, spaced apart locations.

4. The apparatus of claim 1 wherein the magnification marker is carried in a pocket formed in the strap.

5. The apparatus of claim 4 wherein the pocket is openable and closable to permit removal and replacement of the magnification marker.

6. The apparatus of claim 5 wherein the strap is formed with a plurality of pockets for carrying the magnification marker.

7. The apparatus of claim 1 further comprising a layer of wrap material wrapped around the subject between the subject and the strap.

8. The apparatus of claim 1 wherein the strap and fasteners are configured so that the fastened strap moves with the subject region to be radiographed when that region is moved.

9. The apparatus of claim 1 wherein the magnification marker is carried on one end of the strap.

10. A method for securing a magnification marker to a subject having a region that is to be radiographed, comprising the steps of:
positioning an elongated strap to surround the subject around the region to be radiographed;
attaching a radiopaque magnification marker to the strap; and
fastening the strap to itself to define a fastened configuration surrounding the subject, thereby securing the strap to the subject with the magnification marker adjacent to the region to be radiographed.

11. The method of claim 10 including the steps of stretching and sliding the fastened strap relative to the region to adjust the position of the magnification marker relative to the region.

12. The method of claim 11 including the step of locating fasteners on the strap at discrete, spaced apart locations.

13. The method of claim 10 wherein the attaching step comprises placing the magnification marker in a pocket that is formed on the strap.

14. The method of claim 13 including the step of forming the pocket to be openable and closable to permit removal and replacement of the magnification marker.

15. The method of claim 13 including the step of forming the strap to have a plurality of pockets, each pocket being capable of carrying the magnification marker.

16. The method of claim 10 including the step of wrapping the subject with a layer of material prior to positioning the elongated strap to surround the subject around the region.

17. The method of claim 10 further comprising the step of fastening the strap to itself with the strap in a stretched, fastened configuration surrounding the subject so that the fastened strap moves with the subject region to be radiographed when that region is moved.

18. The method of claim 10 further comprising the step of attaching the magnification marker to the strap on one end of the strap.

19. The apparatus of claim 1 including radiopaque indicium carried on the strap or magnification marker.

20. The method of claim 10 further comprising the step of applying radiopaque indicium to the strap or magnification marker.
